(19) United States
(12) Patent Application Publication
(10) Pub. No.: US 2004/0122409 A1
(43) Pub. Date: Jun. 24, 2004

(54) Enhanced elastomer blend

(76) Inventors: Oomman Painumoottil Thomas, Alpharetta, GA (US); David M. Matela, Alpharetta, GA (US); Rasha Wafik Zaki Guirguis, Alpharatta, GA (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/328,856

(22) Filed: Dec. 23, 2002

(51) Int. Cl. A61F 13/15; D02G 3/00

(52) U.S. Cl. 604/385.24; 428/373

(57) ABSTRACT

An elastic blend is made from at least two incompatible polymers and a compatibilizer selected to improve miscibility of the incompatible polymers. For example, a blend of olefinic plastomers and elastomeric styrenic block copolymers, and a compatibilizer having components of the olefinic plastomer and sytrenic block copolymer elastomer yields an inexpensive elastic blend material with adequate elastic properties for use in personal care products.

### BACKGROUND OF THE INVENTION

Personal care products including diapers and sanitary pads often are made with a top sheet material (also referred to as a cover sheet or liner), an absorbent core which is the primary liquid retention layer, and a liquid impervious back sheet, or outer layer. Some such items may also have a surge layer for fluid uptake and distribution, or other specialized layers between the top sheet and absorbent core, and additional gasketing, or containment, flaps within the product. Absorption and retention of fluid, comfort, and avoidance of leakage are the functions desired of such products. Thus, garments often include elasticized portions to create a gasket-like fit around certain openings, such as waist openings and leg openings.

Laminates made from conventional elastic filaments and elastic attachment adhesive are often used to create such elasticized portions. However, such laminates can feel rough or otherwise be uncomfortable. For example, such laminates may cause red-marking on a wearer's skin if the fit is too tight, i.e., elastic tension is too high. Some laminates may result in leakage from the garment if the fit is too loose, i.e., elastic tension is too low. Some elastics may display noticeable tension decay or may become rigid and therefore negatively affect the softness and pliability of the elastic areas of the product, thereby leading to a loss of performance or aesthetics, or both.

There has been a desire in the art to make absorbent garments, such as diapers, better fitting, i.e., more closely conform to the shape of the wearer. One technique for rendering a better fit is to have at least some of the functional layers, e.g., the top and back sheets, expandable, especially laterally or transversely, in the waist area of the garment. It is known in the art that expandability of the garment can be limited by the least expandable layer when said layers are connected in the constructed garment.

Known components for limited use absorbent garments and the like include single site catalyzed polymers such as metallocene catalyzed polymers including metallocene catalyzed polyolefins, e.g., ethylene, propylene, or other olefinic molecules. Examples of such single site catalyzed polymers are available under the tradename AFFINITY from Dow Chemical Co. of Midland Mich., or others. Styrenic block copolymer materials, based on butadiene or isoprene or their hydrogenated or partially hydrogenated versions, are also used, such as are available under the tradename KRATON from Kraton Polymers of Houston, Tex., or others.

Either of these known types of polymers alone may offer challenges for the manufacture of limited use personal products. For example, the extension and retraction properties of single site catalyzed polymers such as metallocene catalyzed polymers are closer to a plastomer than an elastomer, i.e., they are extensible but without great retraction, and therefore are sometimes not adequately elastic for use in all product applications, especially where very high elongation is required. Styrenic block copolymers, while exhibiting more nearly elastomeric extension and retraction properties than metallocene catalyzed polymers, can be expensive for incorporation into limited use personal products. A combination of the two components would be desirable, especially where the combination uses less of the costly styrenic block copolymers. Examples of such blends are disclosed in U.S. Pat. No. 5,853,881 to Estey et al. However, the blends disclosed in Estey et al. provide for a high percentage of the more expensive styrenic block copolymer.

There is a further need or desire for a garment utilizing elastic laminates so as to create elasticized portions of the garment, wherein the elastic has suitable tension properties and is economical for use in a limited use garment resulting in a garment of improved performance or aesthetics, or both.

### SUMMARY OF THE INVENTION

This invention is directed in some aspects to blends of polymers commonly used in personal care products by using a compatibilizer sufficient to create an elastic blend with desired elastic characteristics. Particularly, an elastic blend of the present invention may be optimized for use in personal products such as absorbent garments having elastic blend materials including elastic films or elastic filaments which improve the elastic properties of the material.

In response to the discussed difficulties and problems encountered in the prior art, new elastic blends, and laminates or garments utilizing the new elastic blends, have been discovered. In certain aspects of the present invention, any garment opening such as a waist opening, sleeve or leg cuffs, or necklines may benefit from being made elastic or having elastic components added thereto to improve the fit, hereinafter referred to as “elasticized.” The margins of any garment opening may hereinafter be collectively referred to as “cuffs” or “cuff areas.” Certain aspects of the present invention may provide any one of an elasticized cuff area, non-cuff area, or a containment flap, having extensibility and elasticity for improved fit and the reduced leakage of exudates from an absorbent personal product.

It is desired that personal products, e.g., absorbent articles and garments, and especially garments such as diapers, training pants or incontinence garments, provide a close, comfortable fit about the body of the wearer and contain body exudates while maintaining skin health. In certain circumstances, it is also desirable that such garments are capable of being pulled up or down over the hips of the wearer to allow the wearer or care giver to easily pull the article on and easily remove the article. Other garment openings such as sleeve or pant cuffs and necklines may benefit from being similarly elasticized.

One way of measuring how well elastic materials perform is by measuring their hysteresis. Hysteresis, as used herein, is a measure of how well an elastic material retains its elastic properties between extension and retraction. A sample is cycled from zero elongation to, e.g., 100% elongation and back to zero elongation. A material with no hysteresis would show the same force measured at, e.g., 30 percent elongation during the retraction, or second, half-cycle as the force of extension at 30 percent elongation during the elongation, or first, half-cycle. Percentage of hysteresis may be obtained by subtracting the second half-cycle force of retraction from the first half-cycle force of extension and dividing this number by the first half-cycle force of extension (both at 30 percent elongation, e.g., during a 100% extension/retraction cycle) and multiplying by 100. A material with no difference in force between the extension and retraction half-cycles would have a zero percent hysteresis. A material with some hysteresis would have a hysteresis percentage number above zero. Smaller percentage hysteresis is considered better for present purposes.

In certain aspects of the present invention expandable polyolefin plastomers, e.g., single site catalyzed polyolefins such as metallocene catalyzed polyethylene such as, e.g., commercially available under the trade name AFFINITY from Dow Chemical of Midland, Mich., or other polyolefin plastomers known in the art including polypropylene based plastomers or others; and styrenic block copolymers, such as, e.g., KRATON available commercially from Kraton Polymers, of Houston Tex., are blended together using a compatibilizer such as an ethylene styrene interpolymer such as disclosed in patent publication WO 02/26,882 published Apr. 4, 2002 in the names of Chang, et al, or other interpolymers. Desirably, an elastic blend may include a styrenic block copolymer in the amount of about 30% by weight, a single site catalyzed polyolefin in the amount of about 30% by weight, and a compatibilizer of random or nearly random copolymers, in the amount of about 40% by weight, and having the monomers used in the styrenic block copolymer and the single site catalyzed polyolefin. The solubility parameters of the constituent polymers are selected to improve miscibility and may be about 8.0 to about 9.0 ((cal/cc)^{0.5}); and desirably within about ±1.0 ((cal/cc)^{0.5}) of each other, desirably about ±0.5 ((cal/cc)^{0.5}), more desirably within about ±0.3 ((cal/cc)^{0.5}) of each other, and more desirably substantially the same as each other. Such an elastic blend according to aspects of the present invention is believed to offer adequate elastic performance in a variety of personal product applications at an economical price owing to lesser use of the more expensive styrenic block copolymers.

The compatibilizer chemistry may be reformulated to change the solubility or compatibilizing ability, and may be reformulated to give the resultant blend material desired loading and unloading, also referred to herein as expansion and retraction or stretch and recovery, tensions for personal care product applications. The blend material may be made into filaments or elastic webs and utilized in laminates with other filaments, webs, or films which can be incorporated into personal care products to provide expandable areas such as elastic cuff areas or other areas for garments to improve the elastic characteristics of such areas thereby providing adequate aesthetics and performance for such garments.

Aspects of the present invention are directed to garments utilizing elastic blends, and laminates incorporating such elastic blends, to provide adequate elastic properties. The elastic blend laminates utilized in certain aspects of the invention can be utilized in various combinations of, e.g., a nonwoven facing or facings and elastic filaments, ribbons, or films. A layer of spunbond or other facing material can be laminated along one, or both, surfaces of the film to provide the elastic blend laminates of the invention. Alternatively, it is envisioned that laminates according to the present invention may be produced utilizing the elastic filaments or films placed between primary garment layers such as the back sheet, or outer cover, and liner of the garment. A combination of elastomeric filaments and films might also be suitably used.

The elastic blend may be formulated to provide a variety of materials of differing tension properties. For example the rate and extent of tension, and hysteresis characteristics between the expansion and contraction, may be readily varied according to the dictates of the material application within the product.

### BRIEF DESCRIPTION OF THE DRAWINGS

The accompanying drawings are presented as an aid to explanation and understanding of various aspects of the present invention only and are not to be taken as limiting the present invention.

FIG. 1 illustrates a first personal product according to one aspect of the present invention, in this case an exemplary diaper.

FIG. 2 is a cross sectional view of an elastic laminate according to one aspect of the present invention.

FIG. 3 is a cross sectional view of an alternative elastic laminate according to one aspect of the present invention.

FIG. 4 is a cross sectional view of an alternative elastic laminate according to one aspect of the present invention.

FIGS. 5-14 are graphs showing various elastic performance characteristics of a ternary elastic blend according to the present invention and the components of the ternary blend by themselves.

FIG. 15 illustrates a process for the making of laminates according to one aspect of the present invention.

### DEFINITIONS

Within the context of this specification, each term or phrase below will include the following meaning or meanings.

“Bonded” refers to the joining, adhering, connecting, attaching, or the like, of at least two elements. Two elements will be considered to be bonded together when they are bonded directly to one another or indirectly to one another, such as when each is directly bonded to intermediate elements.

As used herein, the term “consisting essentially of” does not exclude the presence of additional materials which do not significantly affect the desired characteristics of a given composition or product. Exemplary materials of this sort would include, without limitation, pigments, antioxidants, stabilizers, surfactants, waxes, flow promoters, solvents, particulates, and materials added to enhance processability of the composition.

“Denier” refers to a measure of the linear density of fibers in grams per 9000 meters of fiber.

“Elastic blend” refers to an elastic material which is a blend of two or more polymers.

“Elastic tension” refers to the amount of force per unit cross sectional area required to stretch an elastic material, or a selected zone thereof, to a given percent elongation.

“Elastomeric” and “elastic” are sometimes used interchangeably to refer to a material or composite which can be elongated by at least 50 percent of its relaxed length and which will recover with force, upon release of the deformation stress, at least 40 percent of its elongation. It is generally desirable that an elastomeric material or composite be capable of being elongated under low stress by at least 100 percent, more preferably by at least 300 percent, of its relaxed length and recover with force, upon immediate release of the deformation stress, at least 50 percent of its elongation.

An “elastomer” is an elastic polymer. A “plastomer” is an extendable polymer. Polymers which are capable of stretching several times their original dimension when a force is applied and then quickly recover or regain the original dimension or nearly the original dimension when the force is removed are known to exhibit rubber elastic behavior. Polymers which are capable of deformation under the influence of a force but have little or no tendency to regain shape upon the removal of the force are plastic. Plastomers are neither fully elastic nor plastic but show varying degree of elasticity and plasticity under given conditions. Hence, some of their properties, for instance stress-elongation, may appear to be elastic. A plastomer may show 1000%, or 800% or 600% elongation at break. It may give low modulus in the range of 1000 to 7000 psi. however, in certain tests such as hysteresis and tension set, as the elongation becomes higher and higher, a plastomer will show plastic like behavior with a high percentage set and hysteresis while an elastomer in a similar condition gives a low percentage set, and hysteresis.

“Elongation”, refers to the capability of a material to be stretched a certain distance, such that greater elongation refers to a material capable of being stretched a greater distance than a material having lower elongation. “Extensibility” and “expandability” will generally be considered as having the same meaning and may refer to a material property of elongation which does not necessarily recover its shape.

“Film” refers to a thermoplastic film made using a film extrusion process, such as a cast film or blown film extrusion process. The term may include apertured films, slit films, and other porous films which constitute liquid transfer films, as well as films which do not transfer liquid.

“Garment” includes personal care garments, medical garments, and the like. The term “medical garment” includes medical (e.g., protective and/or surgical) gowns, caps, gloves, drapes, face masks, and the like. The term “industrial workwear garment” includes laboratory coats, cover-alls, and the like.

“Incorporate” and “blend” refer to the process of combining two or more elements into a single structure intended to be inseparable.

“Layer” when used in the singular can have the dual meaning of a single element or a plurality of elements.

The terms “limited use” and “disposable” when used in association with personal care products include products which are typically and economically disposed of after 1-5 uses and are not intended to be laundered.

As used herein, the term “machine direction” means the length of a fabric in the direction in which it is produced. The term “cross direction” or “cross machine direction” means the width of fabric, i.e., a direction generally perpendicular to the machine direction.

“Meltblown fiber” refers to fibers formed by extruding a molten thermoplastic material through a plurality of fine, usually circular, die capillaries as molten threads or filaments into converging high velocity gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al., which is incorporated herein in its entirety by reference. Meltblown fibers are microfibers which may be continuous or discontinuous, are generally smaller than about 0.6 denier, and are generally self bonding when deposited onto a collecting surface.

As used herein, the term “neck” or “neck stretch” interchangeably means that the fabric is extended under conditions reducing its width or its transverse dimension. The controlled extension may take place under cool temperatures, room temperature or greater temperatures and is limited to an increase in overall dimension in the direction being extended up to the elongation required to break the fabric. The necking process typically involves unwinding a sheet from a supply roll and passing it through a brake nip roll assembly driven at a given linear speed. A take-up roll or nip, operating at a linear speed higher than the brake nip roll, extends the fabric and generates the tension needed to elongate and neck the fabric. U.S. Pat. No. 4,965,122, to Morman, and U.S. Pat. No. 5,336,545 which are incorporated herein in their entirety by reference, disclose processes for providing a necked nonwoven material laminates.

As used herein, the term “neckable material or layer” means any material which can be necked such as a nonwoven, woven, or knitted material. As used herein, the term “necked material” refers to any material which has been extended in at least one dimension, (e.g. lengthwise), reducing the transverse dimension, (e.g. width), such that when the extending force is removed, the material can be pulled back, or relax, to its original width. The necked material typically has a higher basis weight per unit area than the un-necked material. When the necked material returns to its original un-necked width, it should have about the same basis weight as the un-necked material. This differs from stretching/orienting a material layer, during which the layer is thinned and the basis weight is permanently reduced.

Typically, such necked nonwoven fabric materials are capable of being necked up to about 80 percent, desirably from about 20 to about 60 percent, and more desirably from about 30 to about 50 percent for improved performance. For the purposes of the present disclosure, the term “percent necked” or “percent neckdown” refers to a ratio or percentage determined by measuring the difference between the pre-necked dimension and the necked dimension of a neckable material, and then dividing that difference by the pre-necked dimension of the neckable material and multiplying by 100 for percentage. The percentage of necking (percent neck) can be determined in accordance with the description in the above-mentioned U.S. Pat. No. 4,965,122.

“Nonwoven” and “nonwoven web” refer to materials and webs of material having a structure of individual fibers, or filaments, which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

“Personal products” shall include: absorbent articles used to absorb any fluid including human body fluids, such as diapers, adult incontinence garments, training pants, absorbent swim pants, feminine care products, hygienic wipes, absorbent pads and the like; disposable apparel for institutional, industrial and consumer use; disposable health care products that are not intended to be cleaned for reuse, such as caps, gowns, foot wear, masks, drapes, wraps, covers, and the like; consumer health care products; and health care or environmental diagnostic devices that are at least partially disposable.

“Polymers” include, but are not limited to, homopolymers, copolymers, such as for example, block, graft, random, interpolymers, and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

“Random or nearly random copolymers” as used herein refer to copolymers having the distribution of their (co)monomer repeat units sequence strictly governed by probability, subject only to the relative concentration of the two moieties. The terms shall include interpolymers. Hence, the properties of a random copolymer tend to be an average of the properties of the individual monomers present and are proportional to the relative concentration of the constituent monomers.

“Spunbond fiber” refers to small diameter fibers which are formed by extruding molten thermoplastic material as filaments from a plurality of fine capillaries of a spinneret having a circular or other configuration, with the diameter of the extruded filaments then being rapidly reduced as taught, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Spunbond fibers are quenched and generally not tacky when they are deposited onto a collecting surface. Spunbond fibers are generally continuous and often have average deniers larger than about 0.3, more particularly, between about 0.6 and 10.

“Thermoplastic” describes a material that softens when exposed to heat and which substantially returns to a nonsoftened condition when cooled to room temperature.

“Vertical filament stretch-bonded lamination” or “VF SBL” refers to a stretch-bonded lamination process using a continuous vertical filament process.

Words of degree, such as “about”, “substantially”, and the like are used herein in the sense of “at, or nearly at, when given the manufacturing, design, and material tolerances inherent in the stated circumstances” and are used to prevent the unscrupulous infringer from unfairly taking advantage of the invention disclosure where exact or absolute figures are stated as an aid to understanding the invention.

These terms may be defined with additional language in the remaining portions of the specification.

### DETAILED DESCRIPTION

The various aspects and embodiments of the invention will be described in the context of disposable absorbent articles, and more particularly referred to, without limitation and by way of illustration only, as a disposable diaper. It is, however, readily apparent that the present invention could also be employed to produce other products or garments, such as feminine care articles, various incontinence garments, medical garments and any other disposable garments. Typically, the disposable garments are intended for limited use and are not intended to be laundered or otherwise cleaned for reuse. A disposable diaper, for example, is economically discarded after it has become soiled by the wearer.

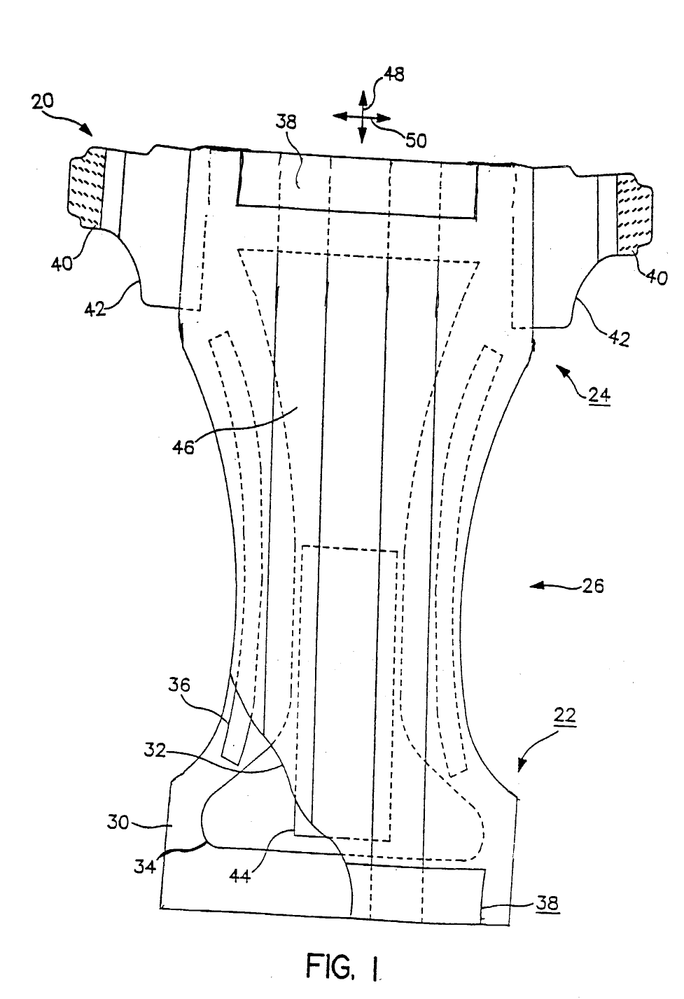

FIG. 1 is a representative plan view of an absorbent article, such as disposable diaper 20, in its flat-out, or unfolded state. Portions of the structure are partially cut away to more clearly show the interior construction of diaper 20. The surface of the diaper 20 which contacts the wearer is facing the viewer.

With reference to FIG. 1, the disposable diaper 20 generally defines a front waist section 22, a rear waist section 24, and an intermediate section 26 which interconnects the front and rear waist sections. The front and rear waist sections 22 and 24 include the general portions of the diaper which are constructed to extend substantially over the wearer's front and rear abdominal regions, respectively, during use. The intermediate section 26 of the diaper includes the general portion of the diaper that is constructed to extend through the wearer's crotch region between the legs.

The diaper 20 includes, without limitation, an outer cover, or back sheet 30, a liquid permeable bodyside liner, or top sheet, 32 positioned in facing relation with the back sheet 30, and an absorbent core, or body, being the primary liquid retention structure, 34, such as an absorbent pad, which is located between the back sheet 30 and the top sheet 32. The back sheet 30 defines a length, or longitudinal direction 48, and a width, or lateral direction 50 which, in the illustrated embodiment, coincide with the length and width of the diaper 20. The liquid retention structure 34 generally has a length and width that are less than the length and width of the back sheet 30, respectively. Thus, marginal portions of the diaper 20, such as marginal sections of the back sheet 30, may extend past the terminal edges of the liquid retention structure 34. In the illustrated embodiment, for example, the back sheet 30 extends outwardly beyond the terminal marginal edges of the liquid retention structure 34 to form side margins and end margins of the diaper 20. The top sheet 32 is generally coextensive with the back sheet 30 but may optionally cover an area which is larger or smaller than the area of the back sheet 30, as desired.

The diaper 20 may include leg elastics 36 which are constructed to operably tension the side margins of the diaper 20 to provide elasticized leg bands which can closely fit around the legs of the wearer to reduce leakage and provide improved comfort and appearance. Waist elastics 38 are employed to elasticize the end margins of the diaper 20 to provide elasticized waistbands. The waist elastics 38 are configured to provide a resilient, comfortably close fit around the waist of the wearer. The person having ordinary skill in the art will appreciate that other areas, such as the front waist section 22, or the entire area of the diaper 20 such as covered by top sheet 32, may be made expandable. Any expandable areas of the diaper 20 may utilize the elastics or laminates as described herein.

In the illustrated embodiment, the diaper 20 includes a pair of side panels 42 to which fasteners 40, indicated as the hook portion of a hook and loop fastener, are attached. Generally, the side panels 42 are attached to the side edges of the diaper 20 in one of the waist sections 22, 24 and extend laterally outward therefrom. The side panels 42 may be expandable. For example, the side panels 42, or indeed, any precursor component webs of the garment, may be a laminate as taught herein and may utilize an elastic or expandable facing material such as a neck-bonded laminate (NBL) or stretch-bonded laminate (SBL) material. Methods of making such materials are well known to those skilled in the art and are described in U.S. Pat. No. 4,663,220 to Wisneski et al., U.S. Pat. No. 5,226,992 to Mornan, U.S. Pat. No. 5,385,775 to Wright, and European Patent Application No. EP 0 217 032 published Apr. 8, 1987 in the names of Taylor et al., each of which is incorporated herein in its entirety by reference. Examples of absorbent articles that include elasticized side panels and selectively configured fastener tabs are described in PCT Patent Application No. WO 95/16425 published Jun. 22, 1995 to Roessler; U.S. Pat. No. 5,399,219 to Roessler et al.; U.S. Pat. No. 5,540,796 to Fries; U.S. Pat. No. 5,595,618 to Fries and U.S. Pat. No. 5,496,298 to Kuepper et al., each of which is incorporated herein in its entirety by reference.

The diaper 20 may also include a surge management layer 44, located between the top sheet 32 and the liquid retention structure 34, to rapidly accept fluid exudates and distribute the fluid exudates to the liquid retention structure 34 within the diaper 20. The diaper 20 may further include a ventilation layer (not illustrated) located between the liquid retention structure 34 and the back sheet 30 to insulate the back sheet 30 from the liquid retention structure 34 to reduce the dampness of the garment at the exterior surface of the back sheet 30. Examples of suitable surge management layers 44 are described in U.S. Pat. No. 5,486,166 to Bishop; U.S. Pat. No. 5,490,846 to Ellis; U.S. Pat. No. 5,364,382 to Latimer et al.; U.S. Pat. No. 5,429,629 to Latimer et al., and U.S. Pat. No. 5,820,973 to Dodge, II et al., each of which is incorporated herein in its entirety by reference.

As representatively illustrated in FIG. 1, the disposable diaper 20 may also include a pair of expandable containment flaps 46 which are configured to provide a barrier to the lateral flow of body exudates. The containment flaps 46 may be located along the laterally opposed side edges of the diaper 20 adjacent the side edges of the liquid retention structure 34. Each containment flap 46 typically defines an unattached edge which is configured to maintain an upright, perpendicular configuration in at least the intermediate section 26 of the diaper 20 to form a seal against the wearer's body.

The present invention incorporates elastic blend materials such as films, ribbons, filaments or webs, and elastic blend laminates having adequate elastic properties for the purposes of personal product manufacture. The blend materials and laminates can be incorporated into any suitable article, such as personal care garments, medical garments, and industrial workwear garments. More particularly, the elastic blend materials and elastic blend material laminates are suitable for use in diapers, training pants, swim wear, absorbent underpants, adult incontinence products, feminine hygiene products, protective medical gowns, surgical medical gowns, caps, gloves, drapes, face masks, laboratory coats, and coveralls.

A number of elastomeric components are known for use in the design and manufacture of such articles. For example, disposable absorbent articles are known to contain expandable and elasticized leg cuffs, elasticized waist portions including cuff areas thereof, elasticized side panels and fastening tabs or other areas. The elastic blend materials and elastic blend material laminates of this invention may be applied to any suitable article to form such expandable and elasticized areas.

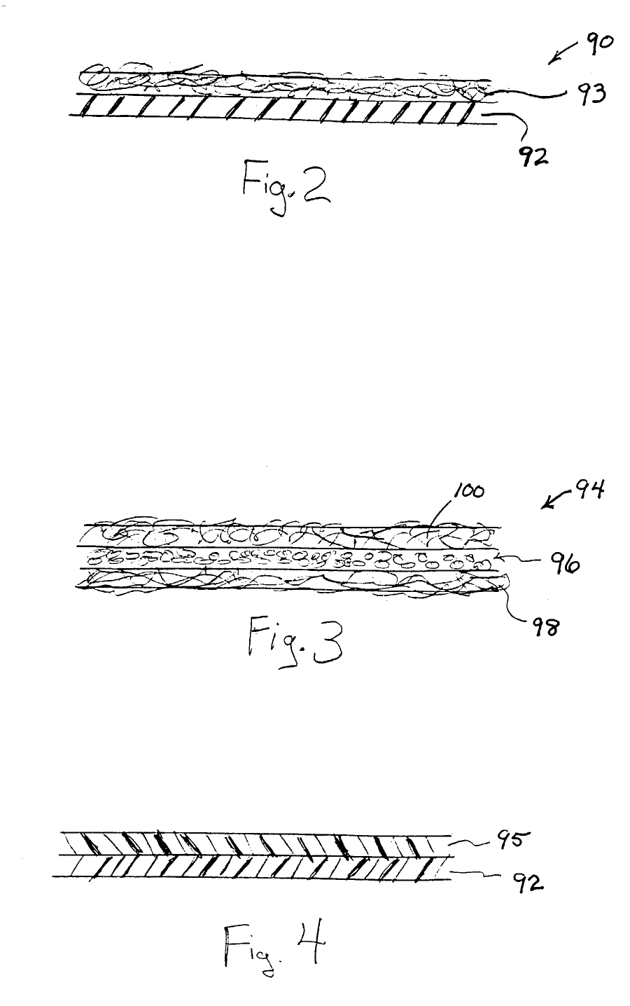

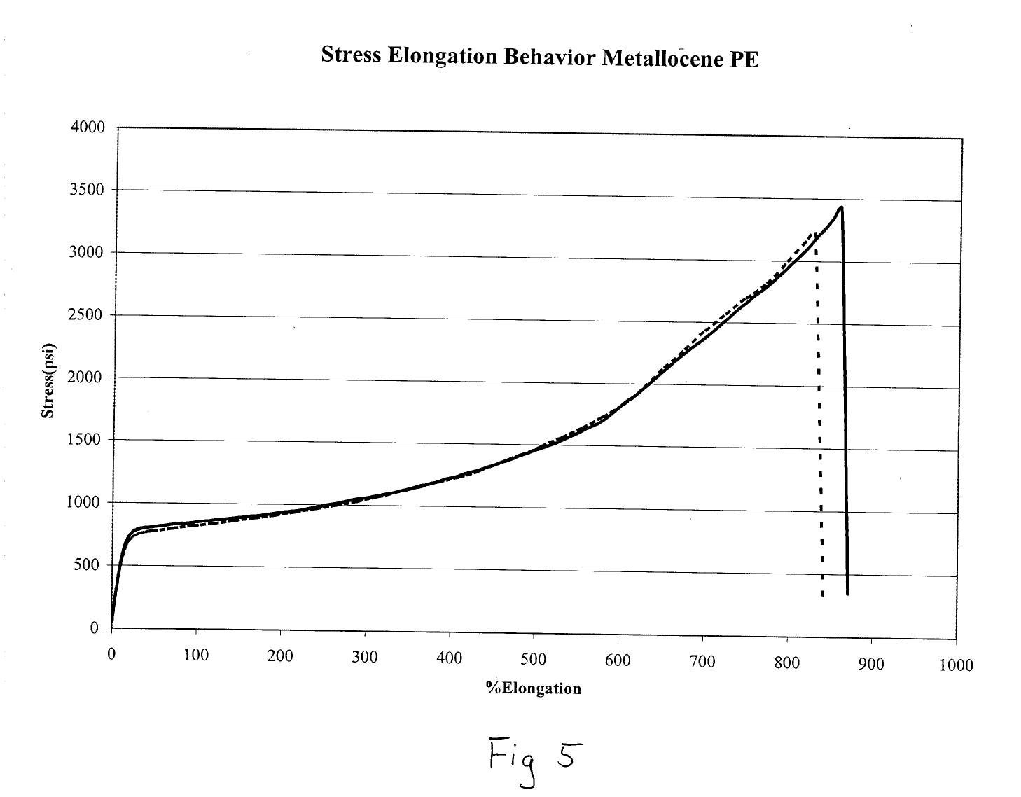

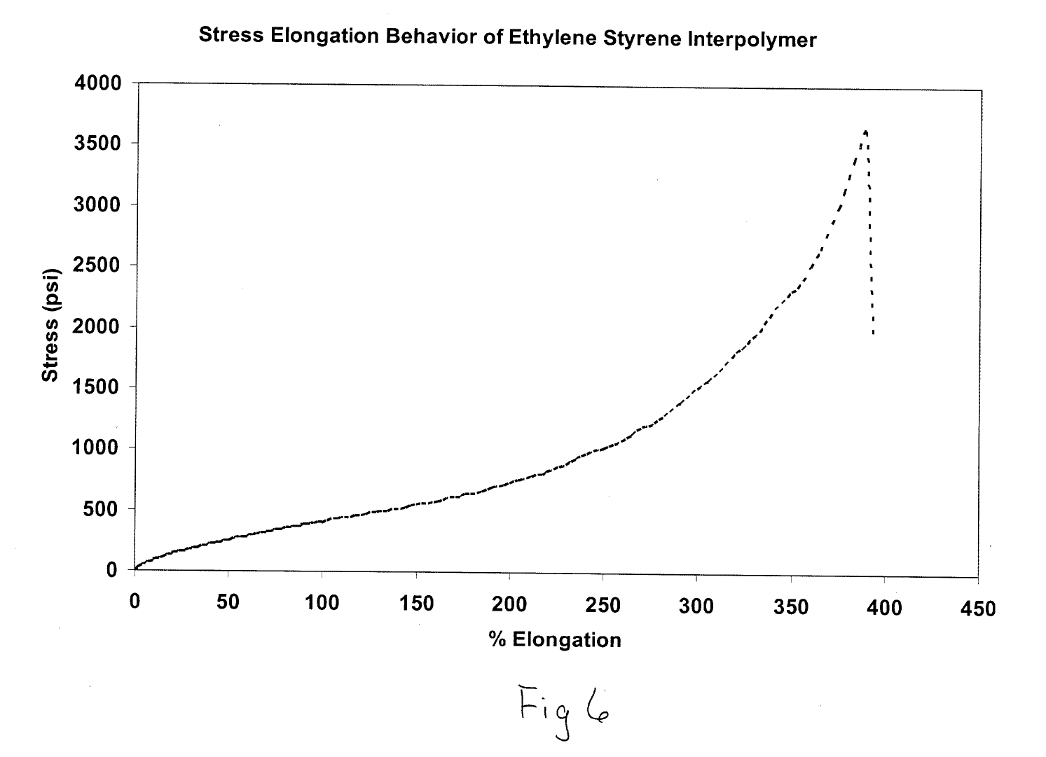

As shown in FIG. 2, an elastic laminate 90 of the invention includes an elastic film 92 with a spun bond nonwoven facing 93. As shown in FIG. 3, an elastic laminate 94 of the invention includes a layer of elastic filaments 96 placed between two nonwoven web facings 98, 100. As seen in FIG. 4, other facings such as films 95 or other nonfibrous webs may be adhered to the elastic blend film 92, or elastic blend filaments 96 in alternative aspects of a laminate according to the present invention.

Suitable blends from which the elastic film 92 may be made include plastomer or elastomer polymers, including sufficient amounts of an elastomeric styrenic block copolymer; a polyolefinic plastomer; and a compatibilizer of a random or nearly random copolymer having components in the styrenic block copolymer and components in the polyolefinic plastomer. The elastic blend may desirably include about 30% by weight of the styrenic block copolymer, about 30% by weight of the metallocene catalyzed polyolefin, and an ethylene/styrene interpolymer compatibilizer in the amount of about 40% by weight. Desirably the solubility parameters of the constituent polymers are selected to improve miscibility and may be between about 8.1 to about 8.7 ((cal/cc)^{0.5}); and within about ±0.5 ((cal/cc)^{0.5}) of each other.

The compatibilizer may comprise an elastic, random, or nearly random copolymer consisting of the monomers used in the styrenic block copolymer and the metallocene catalyzed polyolefin. One such random copolymer is ethylene styrene interpolymer (ESI) as taught in patent publication WO 02/26,882, referenced above. It is also envisioned that propylene styrene interpolymers (PSI) may be suitably formulated according to the present invention where the polyolefin polymers of the blend are polypropylene based. ESI is suitable for use with elastic diblock, triblock, tetrablock, or other multi-block block copolymers including styrene-isoprene-styrene, styrene-butadiene-styrene, styrene-ethylene/butylene-styrene, or styrene-ethylene/propylene-styrene, which may be obtained, e.g., from Kraton Polymers, Inc., under the trade designation KRATON; and polypropylene or polyethylene based plastomers or elastomers including single site catalyzed polyolefins, such as metallocene catalyzed polyolefins commercially available under the tradenames AFFINITY, from Dow Chemical Co. of Midland, Mich., or others, such as may also be known under the name “constrained geometry polyolefins”. The polyolefins may desirably have a density from about 0.80 to 0.95 grams/cubic centimeter (g/cc) and desirably under 0.90 grams/cc, according to some aspects of the invention.

The film of the present invention may generally be a dry-blend processed mixture of a block copolymer such as a styrenic block copolymer, a random or nearly random copolymer (hereinafter collectively referred to for brevity's sake as the “random interpolymer”), and a single site catalyzed polymer, such as a metallocene catalyzed polyolefin polymer, and, if used, any additional components. The film may be made in a dry blend method substantially in accordance with U.S. Pat. No. 6,261,278 to Chen et al., which is hereby incorporated by reference in its entirety. In order to achieve the desired elastic properties for the film of the present invention, it has been discovered that it is desirable that the block copolymer and the single site catalyzed polymer be blended with the random interpolymer ESI such that a ternary blend comprising all of these components is formed. As such, each of the block copolymer and the polyethylene polymer combine to become components of the film. ESI helps to homogenize particle size and distribution of the constituent polymers during the blending. In order to determine the homogeneity or morphology of the blend it is possible to use techniques such as electron microscopy, nuclear magnetic resonance and infrared analysis to evaluate the characteristics of the final, prepared film.

The miscibility of the components and the properties of the resulting blends can further be understood by examining the solubility parameters of the components to be blended. Solubility parameter is based on the square root of the cohesive energy density ((cal/cc)^{0.5}) which is defined as the energy required to remove one molecule form its neighboring molecules. If the solubility parameters of two different polymers to be blended are the same, or about the same, they are expected to be miscible in a thermodynamic sense.

TABLE I
Solubility Parameters, δ (cal/cm³)^{0.5}

| | Poly Styrene | Polyethylene | Polypropylene | Polybutylene |
|---|---|---|---|---|
| *Range | 8.5-9.3 | 7.7-8.4 | 8.2-9.2 | 7.8-8.1 |
| Average | 8.9 | 8.1 | 8.7 | 8.0 |

TABLE II
SepS

| % Styrene | % Ethylene | % Propylene | δ SepS |
|---|---|---|---|
| 10 | 10 | 80 | 8.7 |
| 15 | 15 | 70 | 8.6 |
| 20 | 20 | 60 | 8.6 |
| 25 | 25 | 50 | 8.6 |
| 30 | 30 | 40 | 8.6 |

TABLE III
SebS

| % Styrene | % Ethylene | % Butylene | δ SebS |
|---|---|---|---|
| 10 | 10 | 80 | 8.1 |
| 15 | 15 | 70 | 8.2 |
| 20 | 20 | 60 | 8.2 |
| 25 | 25 | 50 | 8.3 |
| 30 | 30 | 40 | 8.3 |

TABLE IV
ESI

| % Styrene | % Ethylene | δ ESI |
|---|---|---|
| 10 | 90 | 8.2 |
| 20 | 80 | 8.3 |
| 30 | 70 | 8.4 |
| 40 | 60 | 8.4 |
| 50 | 50 | 8.5 |

Table I shows the solubility parameters of various polymers obtained or calculated, and rounded to tenths, according to the reference, D. W. Van Krevelen, “Properties of Polymers,” Elsevier, Amsterdam, 1990. Tables II through IV are solubility parameters calculated for some hypothetical block copolymer or interpolymer compositions according to Table I and using a rule of mixtures approach. The rule of mixtures can be expressed as, solubility parameter, δ=ΦₚₛδₚₛΦₚₑδₚₑ+Φₚₚδₚₚ . . . , where Φ is the weight or volume fraction of a given component and δ is the corresponding solubility parameter, ps=polystyrene, pe=polyethylene, and pp=polypropylene etc. The solubility parameter of a SepS polymer with 20% styrene, 20% ethylene, and 60% propylene would be 8.6 (cal/cm³)^{0.5}. Similar calculations can be made for any given compositions, structures, i.e., ethylene, butylenes, or nature of the blocks, i.e., di, tri, tetra etc. Table IV shows the approximate solubility parameter of several ESI interpolymers, including the 40/60 ESI used in the present examples, which is actually 42% styrene and 58% ethylene. The solubility parameter of the metallocene catalyzed polyethylene plastomer can be considered as that of the polyethylene which is approximately 8.1 ((cal/cc)^{0.5}). Data presented in page 513 of the reference, “Thermoplastic Elastomers,” edited by N. R. Legge, G. Holden, and H. E. Schroeder, and published by Hanser Publishers, Munich, 1987 indicates that the solubility parameters of the hard block (polystyrene) of some of the commercial polymers is approximately 9.9 ((cal/cc)^{0.5}), ethylenepropylene (ep) block 7.7 ((cal/cc)^{0.5}), and ethylenebutylene (eb) block at ˜7.8 ((cal/cc)^{0.5}). These numbers can be used as tools in educing the solubility parameters of different hydrogenated styrenic block copolymers with various styrene and soft block contents.

Although the ultimate miscibility of polymers is dependent on their thermodynamic and kinetic factors which is a function of the chemical structure, molecular weight, and volume fraction etc., solubility parameters can be used as tools in educing the miscibility of different polymers. In order for two polymers to be highly miscible, the difference in their solubility parameter should desirably be close to zero. However, depending on the magnitude of the difference, varying levels of compatibilization are possible and may result in unique morphologies and properties. A general comparison of the calculated values provided in Table II and III with that of IV indicates that the ESI and SepS polymers ought to be more soluble than ESI and SebS polymers. With a solubility parameter of 8.4 ((cal/cc)^{0.5}), ethylene styrene interpolymer appears to compatibilize the metallocene catalyzed polyethylene and the styrenic block copolymers.

In one embodiment of the present invention, after dry mixing together the block copolymer, random interpolymer, and the single site catalyzed polymer to form a dry mixture, such dry mixture is beneficially agitated, stirred, or otherwise blended to effectively uniformly mix the components such that an essentially homogeneous dry mixture is formed. The dry mixture may then be melt blended in, for example, an extruder to effectively uniformly mix the components such that an essentially homogeneous melted mixture is formed. The essentially homogeneous melted mixture may then be used directly, e.g., may be formed into a film or sent directly to other equipment for forming films, or if necessary, cooled and pelletized for later use. Alternative methods of mixing together the components of the present invention include first adding the block copolymer to an extruder and then adding the random interpolymer and the single site catalyzed polymer to such an extruder, wherein the components being used are effectively mixed together within the extruder. In addition, it is also possible to initially melt mix both of the components together at the same time. Other methods of mixing together the components of the present invention are also possible and may be recognized by one skilled in the art.

The process of cooling the extruded thermoplastic composition, in the form of a film, ribbons, or filaments, to ambient temperature is usually achieved by letting the extruded film cool as is or by blowing ambient or sub-ambient temperature air over the extruded film, or extruding onto a chill roll or other controlled temperature roll. For example, the elastic blend may be applied to a chill roll or similar device, in the form of a strand or ribbon. The strand or ribbon can then be stretched and thinned to form the film 92 (FIG. 1). The film suitably has a thickness of about 0.001 inch (1 mil) (0.025 mm) to about 0.05 inch (1.27 mm), alternatively of from about 0.001 inch (0.025 mm) to about 0.01 inch (0.25 mm), and a width of from about 0.05 inch (1.27 mm) to about 3.0 inches (7.62 cm), alternatively of from about 0.5 inch (1.27 cm) to about 15 inches (38.1 cm). The elastic film 92 (FIG. 2) may also be capable of imparting barrier properties in an application.

It is generally desired that the melting or softening temperature of a thermoplastic composition comprising the block copolymer, random interpolymer, and the single site catalyzed polymer be within a range that is typically encountered in most process applications. As such, it is generally desired that the melting or softening temperature of the thermoplastic composition beneficially be between about 25° C. to about 350° C., more beneficially be between about 50° C. to about 300° C., and suitably be between about 60° C. to about 200° C.

It is generally desired that each of the block copolymer, random interpolymer, and the single site catalyzed polymer be melt processable. It is therefore desired that the block copolymer, random interpolymer, and the single site catalyzed polymer used in the present invention each exhibit a melt flow rate that is beneficially between about 1 gram per 10 minutes to about 600 grams per 10 minutes, suitably between about 5 grams per 10 minutes to about 200 grams per 10 minutes, and more suitably between about 10 grams per 10 minutes to about 150 grams per 10 minutes. The melt flow rate of a material may be determined according to a test procedure such as ASTM Test Method D1238-E.

Typical conditions for thermally processing a thermoplastic composition include using a shear rate that is beneficially between about 100 seconds⁻¹ to about 5000 seconds⁻¹ more beneficially between about 500 seconds⁻¹ to about 5000 seconds⁻¹ suitably between about 1000 seconds⁻¹ to about 3000 seconds⁻¹ and most suitably at about 1000 seconds⁻¹. Typical conditions for thermally processing the components also include using a temperature that is beneficially between about 100° C. to about 500° C., more beneficially between about 150° C. to about 300° C., suitably between about 175° C. to about 250° C., and suitably about 200° C. A film of the present invention may generally be of any size or dimension as long as the film exhibits the desired properties as described herein.

The compatibilizer chemistry and moieties, as well as that of the styrenic block copolymer and the metallocene catalyzed polyolefin may be reformulated to vary the characteristics of the resultant elastic blend.

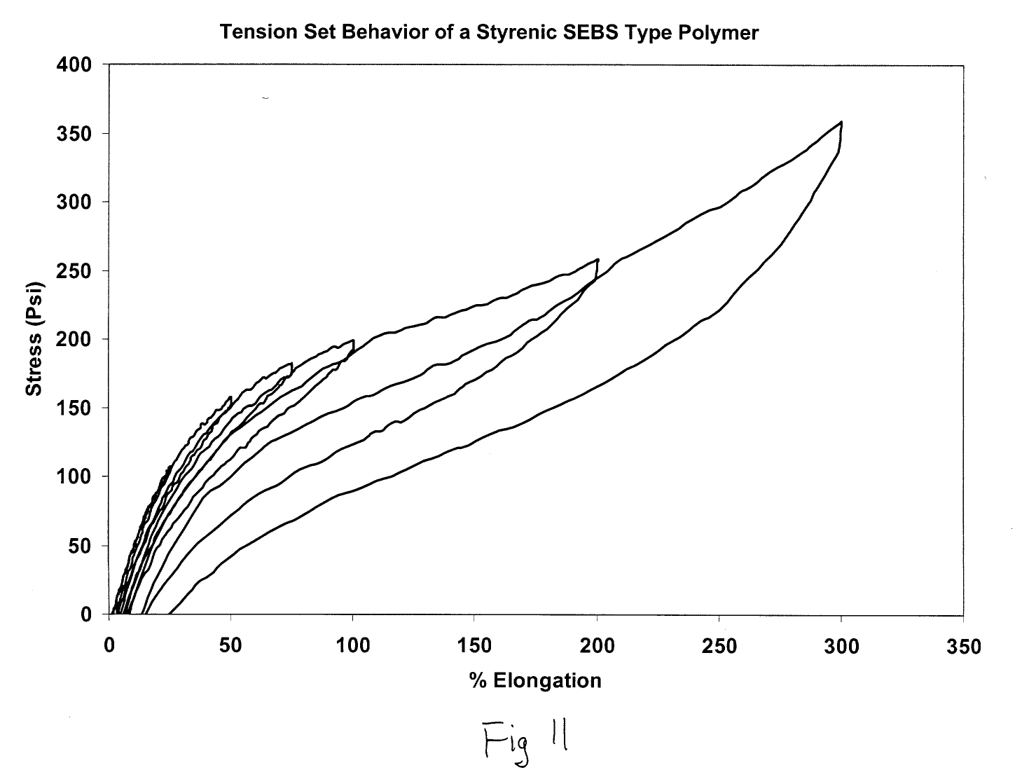

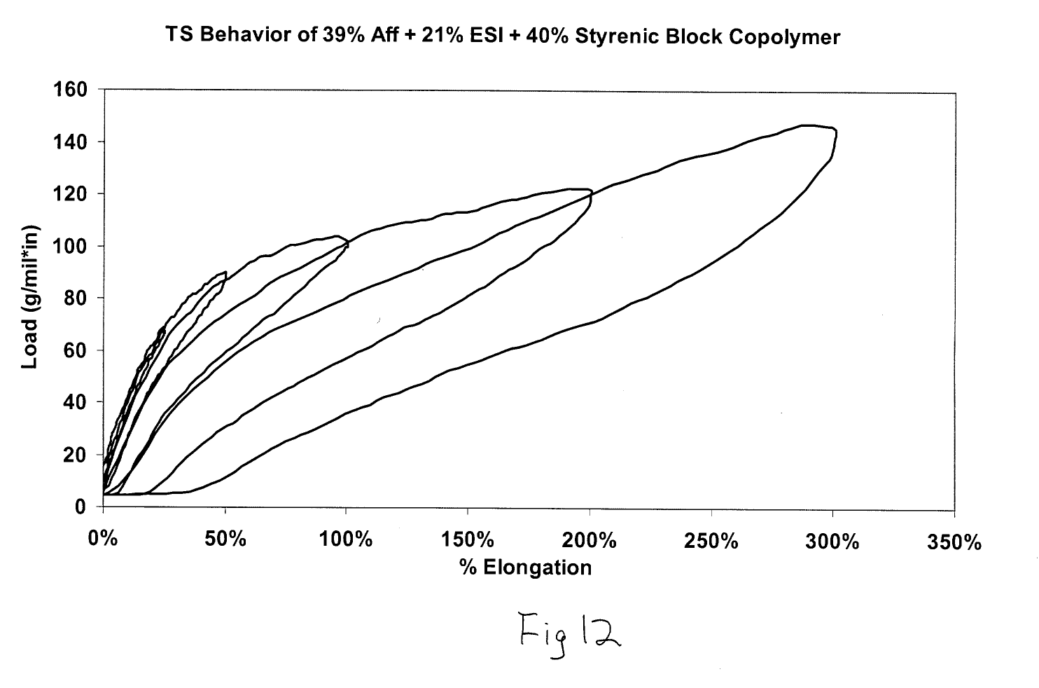

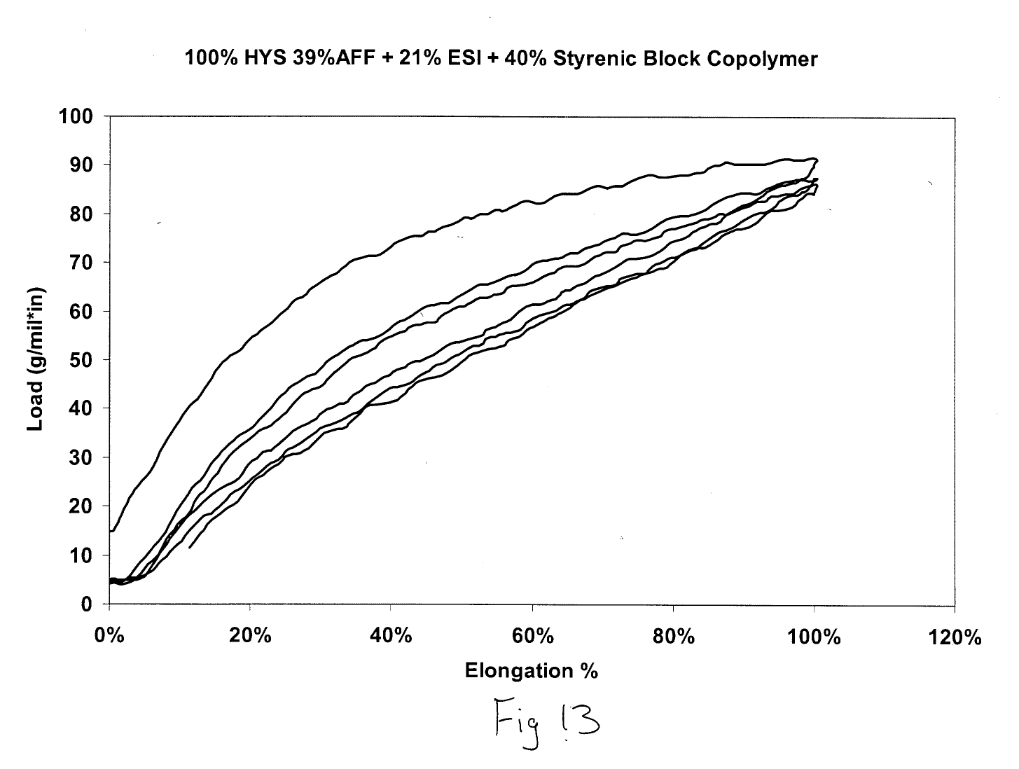

FIG. 5 shows a graph of the stress-elongation behavior of a polyolefin film made using metallocene catalyzed polyethylene, such as the aforementioned AFFINITY type polymers. Film samples for each of the graphs of FIGS. 5-14, were cut in the shape of “dog bone”, approximately 30 mils thick with a center width of 0.5 inches were clamped at a grip-to-grip distance of two inches and were pulled at a cross-head displacement of two inches per minute.

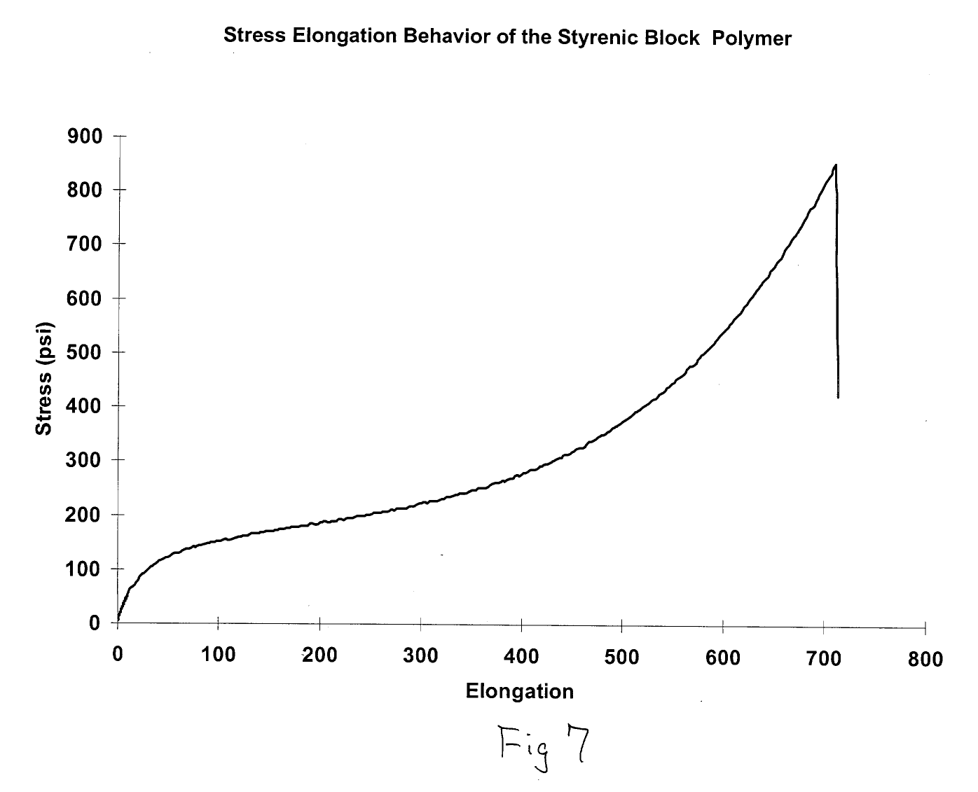

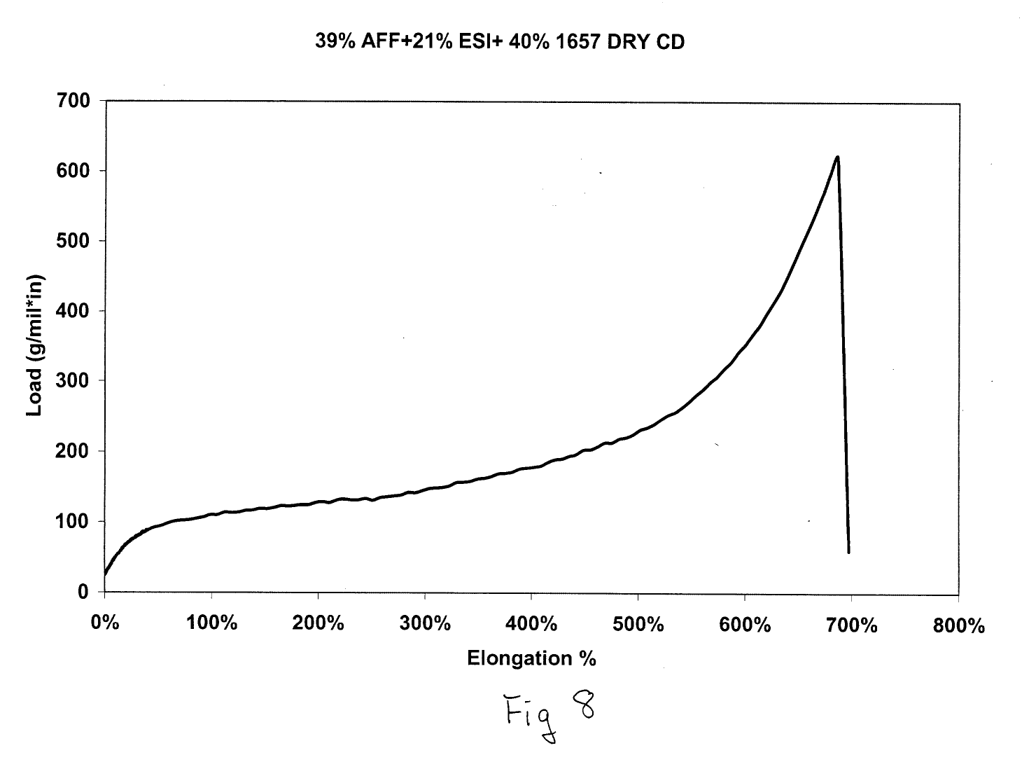

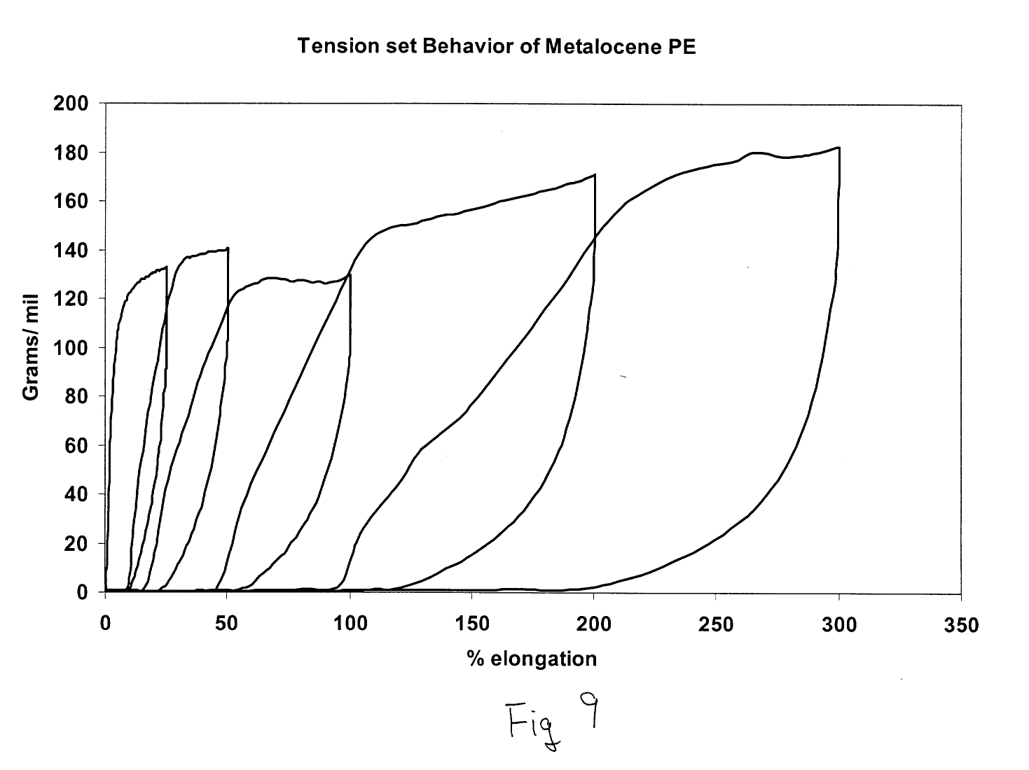

FIG. 6 shows a graph of a typical stress-elongation behavior of a styrenic block copolymer and FIG. 7 shows a graph of a typical stress-elongation behavior of an ethylene-styrene interpolymer. The styrenic block copolymer of FIG. 6 is considered to be a better performing elastomer than the other two polymers.

It can be seen from FIGS. 5-7, as indicated by the initial slope of the stress-elongation curves, that the elastic modulus decreases among these materials in the order of: metallocene catalyzed polyethylene to styrenic block copolymer to ethylene styrene interpolymer. The yield behavior of the metallocene catalyzed polyethylene sample is more extreme than the other two polymers, indicating the more plastic, rather than elastic, character of the metallocene catalyzed polyolefin polymers.

FIG. 8 shows a graph of the stress-elongation behavior of a ternary blend of AFFINITY type metallocene catalyzed polyolefin polymer, ethylene-styrene interpolymer and a styrenic block copolymer known commercially as Kraton G-1657 from Kraton Polymers. Comparisons of FIGS. 5-8 indicate that the ternary blend of FIG. 8 follows the stress-elongation behavior of the styrenic block copolymer (KRATON) of FIG. 7, indicating that the ternary blend is a better performing elastomer than the individual homopolymers of FIGS. 5 and 6.

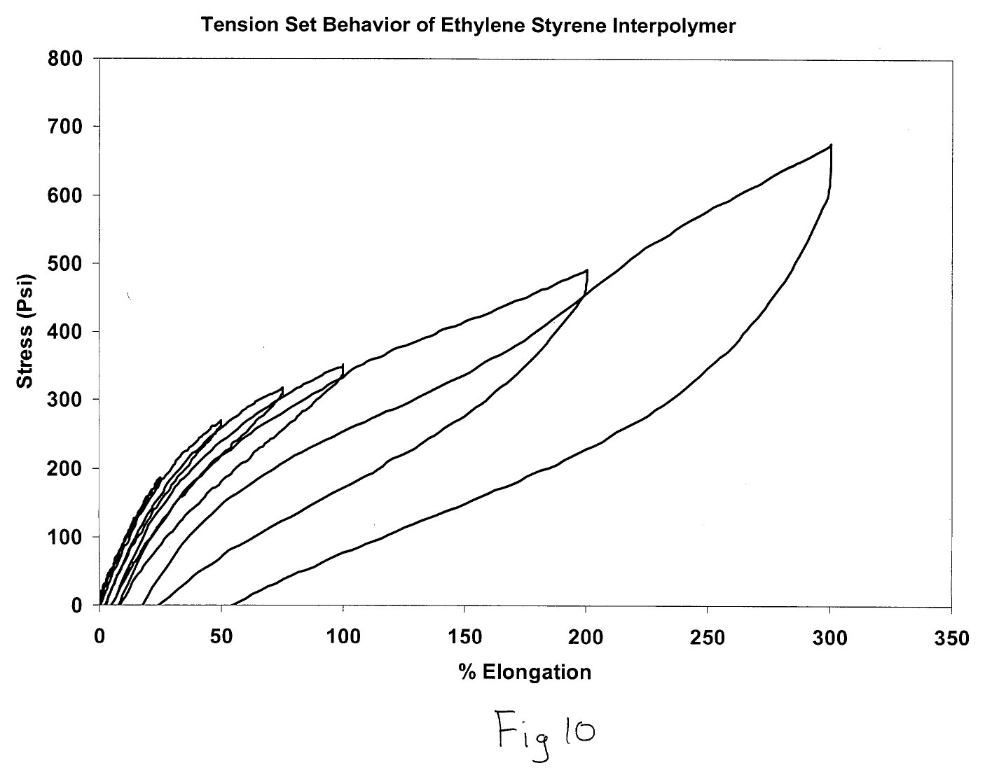

A significant difference can be seen from the tension set data provided in FIGS. 9, 10, 11, and 12. “Tension set” is an intermittent test in which a stress-elongation value is obtained by stretching a sample to a predetermined elongation. The sample is then released and then stretched to a next greater degree of elongation and so on. The elongation at a load corresponding to zero after the removal of the applied elongation is then measured. The tension set is a measure of the irreversibility of deformation.

FIG. 9 shows the tension set behavior of metallocene catalyzed polyethylene and it can be seen that there is very little tendency for the polymer to recover its original shape after each cycle up to 300% cycle elongation. It will be noted that the irrecoverable percentage elongation associated with the 300% cycle for the metallocene catalyzed polyethylene is about 200%. This indicates that this polymer is behaving plastically rather than elastically. The percentage of “set” associated with the 300% elongation cycle of the styrenic block copolymer and ethylene styrene interpolymers are approximately 25% and slightly above 50%. The set values seen in the Fig. indicate an elastic behavior and are substantially better for the present purposes than the metallocene based polymer which is more plastic like. The tension set behavior of the ternary blend is between the ESI and styrenic block copolymer suggesting that the ternary blend components are compatible and thus provide good elastic properties.

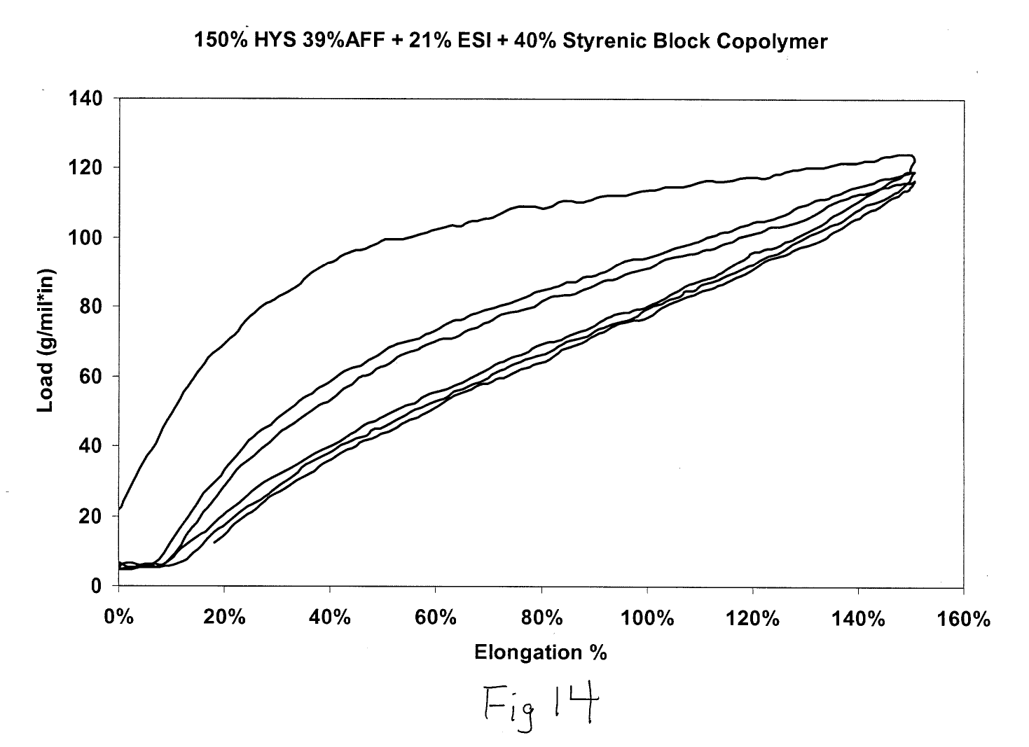

FIGS. 13 and 14 show the 100 and 150% cycle extension and retraction curves for the ternary blend. It can be seen from these figures that as cycle elongation increases from 100% to 150%, the hysteresis increases, as indicated by the area of the loop.

The elastic blend film 92 suitably may have an elongation of at least 50 percent, alternatively of at least 150 percent, alternatively of from about 50 percent to about 200 percent. The elastic blend film 92 may further suitably have a retractive force of less than about 400 grams force per inch (2.54 cm) width, alternatively of less than about 275 grams force per inch (2.54 cm) width, alternatively of from about 100 grams force per inch (2.54 cm) width to about 250 grams force per inch (2.54 cm) width, as determined by a tensile tester at one minute of stretching the film to a ninety percent elongation. Such a tensile tester may be obtained commercially from the Materials Testing Corporation of Minneapolis, Minn., as SINTECH Model No. 11.

The elastic laminates of the invention include the above-described elastics 90 bonded to a first facing sheet, e.g., 93, 98, and a second facing sheet 100 as shown in FIGS. 2, 3 and 4. Facing materials may be formed using conventional processes, including the spunbond and meltblowing processes such as described above. For example, the facing sheets may each include a spunbond web having a basis weight of about 0.1-4.0 ounces per square yard (osy), suitably 0.2-2.0 osy, or about 0.4-0.8 osy. The facing sheets may include the same or similar materials or different materials. Alternatively, it is envisioned that laminates according to the present invention may be produced utilizing the elastic blend material placed between primary garment layers such as the back sheet 30 and top sheet 32 (FIG. 1).

If the facing sheets are to be applied to the elastic blend, the facing sheets may be extensible or non-extensible depending upon their ultimate application in the product. In one aspect of the invention, the facing sheets could be necked, crimped, or gathered, or combinations thereof, in order to allow them to be stretched after application of the elastic blend. Facing layers including a known necked nonwoven facing layer such as 0.6 osy spunbond may be bonded to the elastic blend by adhesives, thermal bonding, ultrasonic bonding or other known methods.

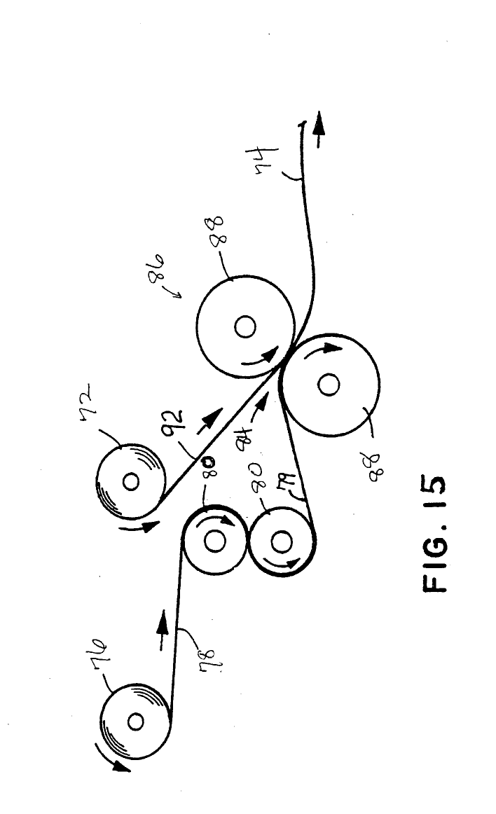

Referencing FIG. 15, a process for making one exemplary laminate 74 according to the present invention is shown. The elastomeric blend material in the form of a film 92 is unwound from a supply roll 72. In order to form the elastic laminate 74, at least one, or a first, roll 76, respectively, of a spunbond facing material 93, such as spunbond nonwoven between about 0.2-2.0 osy having fiber denier of approximately 2.0-2.5, and e.g., containing approximately 50% Polyethylene and 50% Polypropylene in a side-by-side configuration, and thermally point bonded, is fed between tensioning S-rollers, collectively 80 to be initially necked.

The elastic blend material 92 passes through the nip 84 of the bonder roller arrangement 86 formed by the bonder rollers 88 collectively. The initially necked spunbond material 78 then passes through the nip 84 of the bonder roller arrangement 86. Because the peripheral linear speed of the S-rollers 80 is controlled to be less than the peripheral linear speed of the rollers 88 of the bonder roller arrangement 86, the initially necked spunbond material 78 is further tensioned, as at 79, between the S-rollers 80 and the nip 84 of the bonder roll arrangement 86. By adjusting the difference in the speeds of the rollers, the spunbond facing material is tensioned so that it necks a desired amount, e.g. 50%, and is maintained in such tensioned, necked condition while the elastic film material 92 is joined to the necked spunbond material during their passage through the bonder roller arrangement 86 to form a composite elastic necked-bonded laminate 74. It will be appreciated that laminates according to the present invention may be made from non-necked facing materials such as inherently extendable nonwovens such as certain forms of bonded carded web (BCW), inherently elastic material webs, or the like. It will be appreciated that other processes consistent with the present invention may be used such as the aforementioned SBL process, a horizontal lamination process as taught in U.S. Pat. No. 5,385,775 to Wright, or a vertical filament lamination process (VFL) as taught in published US Patent Application No. US2002-0104608, both of which references are hereby incorporated by reference in their entirety; or combinations of known lamination processes.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the exemplary embodiments, yet the absence of a particular advantage shall not be construed to necessarily mean that such an embodiment is outside the scope of the present invention.

We claim:

1. An elastic material comprising a blend of:
a) an elastomeric styrenic block copolymer;
b) a polyolefinic plastomer; and
c) a compatibilizer including components of the styrenic block copolymer and components of the polyolefinic plastomer.

2. The elastic material of claim 1 wherein the polyolefinic plastomer is a single site catalyzed polyethylene.

3. The elastic material of claim 1 wherein the single site catalyzed polyethylene is a metallocene catalyzed polyethylene.

4. The elastic material of claim 1 wherein the polyolefinic plastomer is a polypropylene based plastomer.

5. The elastic material of claim 4 wherein the polyolefinic plastomer is a polypropylene based plastomer with a solubility parameter of between about 8.2 to about 9.2 ((cal/cc)^{0.5}).

6. The elastic material of claim 4 wherein the polypropylene based plastomer has a solubility parameter of about 8.7 ((cal/cc) 0.5).

7. The elastic material of claim 2 wherein the compatibilizer is an ethylene/styrene interpolymer.

8. The elastic material of claim 4 wherein the compatibilizer is a propylene/styrene interpolymer.

9. The elastic material of claim 1 wherein the solubility parameters of the elastomeric styrenic block copolymer; the polyolefinic plastomer; and the compatibilizer are selected to improve miscibility and are about 8.6 ((cal/cc)^{0.5}), about 8.1 ((cal/cc)^{0.5}), and about 8.4 ((cal/cc)^{0.5}), respectively.

10. The elastic material of claim 1 wherein the solubility parameters of the elastomeric styrenic block copolymer; the polyolefinic plastomer; and the compatibilizer are within about ±1.0 ((cal/cc)^{0.5}) of each other.

11. The elastic material of claim 1 wherein the solubility parameters of the elastomeric styrenic block copolymer; the polyolefinic plastomer; and the compatibilizer are within about ±0.3 ((cal/cc)^{0.5}) of each other.

12. An elastic material suitable for use with an extendable layer of an absorbent article, comprising a blend of:
a) a styrenic block copolymer in the amount of about 30% by weight
b) a single site catalyzed polyolefin in the amount of about 30% by weight; and
c) random or nearly random copolymers consisting of the monomers used in the styrenic block copolymer and the single site catalyzed polyolefin, in the amount of about 40% by weight.

13. The elastic material of claim 12 wherein the polyolefinic plastomer is a single site catalyzed polyethylene.

14. The elastic material of claim 12 wherein the single site catalyzed polyethylene is a metallocene catalyzed polyethylene.

15. The elastic material of claim 13 wherein the compatibilizer is an ethylene/styrene interpolymer.

16. The elastic material of claim 12 wherein the solubility parameters of the elastomeric styrenic block copolymer; the single site catalyzed polyolefin; and the random or nearly random copolymers are within ±0.3 ((cal/cc)^{0.5}) of each other.

17. An elastic laminate, comprising:
a) a layer of an elastic material having a first and a second side;
b) a second layer of material;
c) the layer of elastic material bonded to the second layer of material on at least one of the first and second sides of the elastic material;
d) wherein the elastic material comprises:
i) an elastomeric styrenic block copolymer;
ii) a polyolefinic plastomer; and
iii) a compatibilizer including components of the styrenic block copolymer and components of the polyolefinic plastomer.

18. The laminate of claim 17 wherein the first layer comprises filaments of the elastic material.

19. The laminate of claim 17 wherein the first layer comprises a film of the elastic material.

20. The laminate of claim 17 wherein the second layer comprises a necked nonwoven web.

21. The laminate of claim 17 wherein the second layer comprises an expandable web.

22. The laminate of claim 17 wherein the second layer comprises an elastic film.

23. The laminate of claim 17 wherein the polyolefinic plastomer is a single site catalyzed polyethylene.

24. The laminate of claim 17 wherein the single site catalyzed polyethylene is a metallocene catalyzed polyethylene.

25. The laminate of claim 17 wherein the polyolefinic plastomer is a polypropylene based plastomer.

26. The laminate of claim 25 wherein the polypropylene based plastomer has a solubility parameter of between about 8.2 to about 9.2 ((cal/cc)^{0.5}).

27. The laminate of claim 25 wherein the polypropylene based plastomer has a solubility parameter of about 8.7 ((cal/cc)^{0.5}).

28. The laminate of claim 23 wherein the compatibilizer is an ethylene/styrene interpolymer.

29. The laminate of claim 25 wherein the compatibilizer is a propylene/styrene interpolymer.

30. The laminate of claim 24 wherein the solubility parameters of the elastomeric styrenic block copolymer; the polyolefinic plastomer; and the compatibilizer are selected to improve miscibility and are about 8.6 ((cal/cc)^{0.5}), about 8.1 ((cal/cc)^{0.5}), and about 8.4 ((cal/cc)^{0.5}), respectively.

31. The laminate of claim 17 wherein the solubility parameters of the elastomeric styrenic block copolymer; the polyolefinic plastomer; and the compatibilizer are within about ±1.0 ((cal/cc)^{0.5}) of each other.

32. The laminate of claim 17 wherein the solubility parameters of the elastomeric styrenic block copolymer; the polyolefinic plastomer; and the compatibilizer are within about ±0.3 ((cal/cc)^{0.5}) of each other.

33. A laminate suitable for use with an extendable layer of an absorbent article, comprising:
a) a first layer including a blend of:
i) a styrenic block copolymer in the amount of about 20% to about 40% by weight;
ii) a single site catalyzed polyolefin in the amount of about 20% to about 40% by weight; and
iii) an ethylene/styrene interpolymer in the amount of about 20% to about 60% by weight; and
b) a second layer including a facing layer adhered to the first layer.

34. The laminate of claim 33 wherein the first layer comprises filaments of the blend.

35. The laminate of claim 33 wherein the first layer comprises a film of the blend.

36. The laminate of claim 33 wherein the second layer comprises a necked nonwoven.

37. The laminate of claim 33 wherein the second layer comprises an expandable web.

38. The laminate of claim 33 wherein the second layer comprises an elastic film.

39. An absorbent personal product, comprising:
I) a liner;
II) an outer cover;
III) an absorbent layer between the liner and the outer cover; and
IV) side panels comprising an elastic laminate having:
a) a layer of an elastic material having a first and a second side;
b) a second layer of material;
c) the layer of elastic material bonded to the second layer of material on at least one of the first and second sides of the elastic material;
d) wherein the elastic material comprises:
i) an elastomeric styrenic block copolymer;
ii) a polyolefinic plastomer; and
iii) a compatibilizer including components of the styrenic block copolymer and components of the polyolefinic plastomer.

40. The absorbent personal product of claim 39 wherein the polyolefinic plastomer is a single site catalyzed polyethylene.

41. The absorbent personal product of claim 40 wherein the single site catalyzed polyethylene is a metallocene catalyzed polyethylene.

42. The absorbent personal product of claim 40 wherein the compatibilizer is an ethylene/styrene interpolymer.

43. The absorbent personal product of claim 39 wherein the solubility parameters of the elastomeric styrenic block copolymer; the polyolefinic plastomer; and the compatibilizer are selected to improve miscibility and are about 8.6 ((cal/cc)^{0.5}), about 8.1 ((cal/cc)^{0.5}), and about 8.4 ((cal/cc)^{0.5}), respectively.

44. The absorbent personal product of claim 39 wherein the solubility parameters of the elastomeric styrenic block copolymer; the polyolefinic plastomer; and the compatibilizer are within ±about 0.3 ((cal/cc)^{0.5}) of each other.

45. The absorbent personal product of claim 39 wherein the second layer of material comprises a nonwoven web.

46. The absorbent personal product of claim 39 wherein the outer cover is expandable.

47. The absorbent personal product of claim 39 wherein the outer cover is elastic.

48. A method of making an elastic laminate, comprising:
a) making a blend of polymers comprising:
i) an elastomeric styrenic block copolymer;
ii) a polyolefinic plastomer; and
iii) a compatibilizer including components of the styrenic block copolymer and components of the polyolefinic plastomer;
b) forming the blend into a layer of elastic material having a first and a second side;
c) providing a second layer of material;
d) bonding the layer of elastic material to the second layer of material on at least one of the first and second sides of the elastic material.

49. The method of making an elastic laminate of claim 48 wherein the polyolefinic plastomer is a single site catalyzed polyethylene.

50. The method of making an elastic laminate of claim 49 wherein the single site catalyzed polyethylene is a metallocene catalyzed polyethylene.

51. The method of making an elastic laminate of claim 49 wherein the compatibilizer is an ethylene/styrene interpolymer.

52. The method of making an elastic laminate of claim 48 wherein the solubility parameters of the elastomeric styrenic block copolymer; the polyolefinic plastomer; and the compatibilizer are selected to improve miscibility and are about 8.6 ((cal/cc)^{0.5}), about 8.1 ((cal/cc)^{0.5}), and about 8.4 ((cal/cc)^{0.5}), respectively.

53. The method of making an elastic laminate of claim 48 wherein the solubility parameters of the elastomeric styrenic block copolymer; the polyolefinic plastomer; and the compatibilizer are within ±0.3 ((cal/cc)^{0.5}) of each other.

54. The method of making an elastic laminate of claim 49 wherein:
the styrenic block copolymer is present in the amount of about 30% by weight;
the single site catalyzed polyolefin is present in the amount of about 30% by weight; and
the random or nearly random copolymers are present in the amount of about 40% by weight.
